(19) United States
(12) Patent Application Publication
(10) Pub. No.: US 2022/0040051 A1
(43) Pub. Date: Feb. 10, 2022

(54) COMPOSITION

(71) Applicant: COSMETIC WARRIORS LIMITED, Poole, Dorset (GB)

(72) Inventors: Mark CONSTANTINE, Poole, Dorset (GB); Margaret Joan CONSTANTINE, Poole, Dorset (GB); Helen Elizabeth AMBROSEN, Poole, Dorset (GB); Rowena Jacqueline BIRD, Poole, Dorset (GB); Daniel CAMPBELL, Poole, Dorset (GB)

(21) Appl. No.: 17/276,086

(22) PCT Filed: Sep. 11, 2019
(86) PCT No.: PCT/GB2019/052534

§ 371 Date: Mar. 12, 2021

(30) Foreign Application Priority Data
Sep. 14, 2018 (GB) 1815031.8
Jan. 28, 2019 (GB) 1901161.8

(51) Int. Cl. A61K 8/02; A61K 8/9789; A61K 8/92; A61Q 19/10; A61Q 5/02; A61K 8/46

(52) U.S. Cl. CPC A61K 8/0216; A61K 8/9789; A61K 8/92; A61K 2800/48; A61Q 5/02; A61K 8/463; A61Q 19/10

(57) ABSTRACT

A solid cosmetic composition includes: (i) a surfactant; (ii) a wax; and (iii) aquafaba.

### FIELD OF THE INVENTION

The present invention relates to a solid cosmetic composition, a process for producing said solid cosmetic composition, a product prepared by the process, and a cosmetic method for using the solid cosmetic composition. The present invention also relates to a use of aquafaba.

### BACKGROUND TO THE INVENTION

The present invention relates to solid cosmetic compositions, particularly those for use in contact with the human or animal body.

For millennia soaps have been used for the purposes of washing the skin and clothes. The first known use of soap as a cleansing agent was in the ancient Babylonian period, approximately 2800 BC, where soap formed from water, cinnamon oil and dissolved ashes was used to wash clothes and prepare wool for weaving. Egyptians also substantiated the use of soap derivatives, for the purposes of personal hygiene, medicinal applications and the effective washing of garments.

However it was not until AD77 that Gaius Plinius Secundus (also known as Pliny the Elder) published his encyclopaedic work, Historia Naturalis, whereby the first references to sapo (Latin for soap) were made. References were made to the manufacture of sapo, from tallow and ashes, whilst also describing its potential application as a pomade for hair.

The appeal of soap as a cleansing and personal hygiene agent grew. Although it was not until the 15th and 16th centuries, when soap production became semi-industrialised and the role it played in personal hygiene was understood, that soap became a universally accepted cleansing product in industrialised nations. However it was the First World War that prompted a shift in this trend, which resulted in the development of the first synthetic detergents, in response to a shortage of animal and vegetable fats used for making soap.

It is well known in the art that soap is effectively the salt of a fatty acid, resulting from the saponification of an animal or vegetable triglyceride using a strong base. Whilst soap is known to be an effective cleansing agent, soap can cause dryness and irritation to dry and sensitive skin. This is due to the basicity of soap and its cleansing action adversely affecting the cutaneous bacterial flora, enzyme activity in the upper epidermis and naturally occurring oils found on the skin and hair. Therefore, soap is not generally considered to be suitable for washing the hair of a user as it may leave the hair feeling dry and waxy. Furthermore, particularly when used with hard water, soap may form a soap scum which is not desirable for a user. In particular, soap scum may stick in the user's hair and be difficult to wash out. In addition, it is generally known that a soap bar does not smoothly glide on skin when being applied; rather a soap bar may “drag” on skin, leading to an undesirable sensation for the user.

Synthetic detergents on the other hand can be synthesised in order to exhibit a wide variety of different properties that are particularly beneficial for their particular application. The desired properties of synthetic detergents used as skin or hair cleansers vary hugely from mild, conditioning surfactants to cleansers with high foaming abilities. These synthetic detergents are commonly referred to as surfactant products; for example, shower gels and shampoos. However, these surfactant-containing products are typically provided in the form of liquid or pastes. They are sold in containers to the end user and may be dispensed by the end user. However, the required use of packaging is a disadvantage. From an environmental perspective, waste packaging is a significant problem, despite the availability of recycling.

There is also a desire to provide more ‘exciting’ products which add to the bathing experience of a user. In other words, there is a desire to provide products which offer to the user more than the functional delivery of a surfactant to be able to wash the hair or skin as desired.

The present invention seeks to provide a solid cosmetic product, which is a surfactant-containing product that may be used for washing both the skin and hair of a user.

### SUMMARY OF THE INVENTION

According to a first aspect of the invention, there is provided a solid cosmetic composition comprising (i) a surfactant; (ii) a wax; and (iii) aquafaba.

According to a second aspect of the invention, there is provided a process for the production of a solid cosmetic composition according to the first aspect of the invention, the process comprising the steps of:
(a) combining:
   (i) a surfactant;
   (ii) a wax; and
   (iii) aquafaba; and
(b) solidifying the resultant composition.

According to a third aspect of the invention, there is provided a product obtained or obtainable by a process, the process comprising the steps of:
(a) combining:
   (i) a surfactant;
   (ii) a wax; and
   (iii) aquafaba; and
(b) solidifying the resultant composition.

According to a fourth aspect of the invention, there is provided a cosmetic method comprising contacting the skin or hair of a user with the solid cosmetic composition according to the first aspect of the invention.

The present inventors surprisingly found that the combination of aquafaba with a surfactant and a wax resulted in a product having a texture and structure akin to that of a marshmallow, whilst also being suitable for use in washing the skin and hair of the user. The light marshmallow-like texture of the product provides the user with a more exciting product to wash their hair and body with. The light marshmallow-like texture has also proved to be beneficial as it allows easy transport and storage of the product due to its lightweight structure. A user may therefore be able to store and transport a large volume of the product without undue burden as its low density means that such large volume of product has a relatively low mass.

Indeed, by forming a cosmetic solid product comprising aquafaba, wax and surfactant, a product having a low density may be prepared. In other words for a given volume a cosmetic product may be provided having less mass or in other words containing less cosmetic material. Thus the present invention allows the user to hold a product which is of a suitable size and which also offers an enjoyable massage or easy application to the skin/hair, while containing less cosmetic ingredient thereby minimising waste on disposal or allowing application of all of the product when providing a single massage or application to an individual.

Advantageously, use of aquafaba also enables the production of a cosmetic product having a similar texture to that of marshmallow, whilst being a vegan product. In other words, the use of aquafaba enables the production of a marshmallow-like product that does not require the use of animal-based products, such as eggs or gelatin.

For ease of reference, these and further aspects of the present invention are now discussed under appropriate section headings. However, the teachings under each section are not necessarily limited to each particular section.

### DETAILED DESCRIPTION

### Composition

As described herein, in one aspect of the present invention, there is provided a solid cosmetic composition comprising: (i) a surfactant; (ii) a wax; and (iii) aquafaba.

It will be understood by one skilled in the art that the nature of a cosmetic product means that the product is not edible. Thus, the present invention provides a non-edible solid cosmetic composition as defined herein.

Solid cosmetic products of the present invention are compositions which can substantially sustain their physical shape when unsupported by external means, e.g. packaging etc. Thus, they are considered to be solid, solid-like, in solid form or in solid-like form at room temperature. For the avoidance of doubt, the solid product is one which remains substantially solid at a temperature of up to 30° C.

By solid-like, it is understood that some materials are considered on a day to day basis to be solid, yet over an extremely long period of time, may alter in shape, e.g.

amorphous materials such as glass etc. However, they are considered to be solid-like as, for the purpose they fulfil, they are solid.

As mentioned above, due to the solid form of the compositions of the present invention, external packaging is not required to maintain the shape of the composition.

### Surfactant

As described herein, the solid cosmetic composition comprises a surfactant. As the skilled person will appreciate, surfactants are typically sold as commercial products in which the pure surfactant (i.e. the active surfactant) is either diluted in water or is provided as a solid product (e.g. as solid surfactant needles). As used herein, the amounts of the surfactant do not relate to the commercially available diluted products, but rather to the amount of pure surfactant.

In some embodiments, the surfactant is present in an amount of from about 15% to about 60% by weight of the solid cosmetic composition. In some embodiments, the surfactant is present in an amount of from about 20% to about 60% by weight of the solid cosmetic composition. In some embodiments, the surfactant is present in an amount of from about 25% to about 55% by weight of the solid cosmetic composition. In some embodiments, the surfactant is present in an amount of from about 25% to about 50% by weight of the solid cosmetic composition. In some embodiments, the surfactant is present in an amount of from about 30% to about 50% by weight of the solid cosmetic composition. In some embodiments, the surfactant is present in an amount of from about 30% to about 45% by weight of the solid cosmetic composition. In some embodiments, the surfactant is present in an amount of from about 30% to about 40% by weight of the solid cosmetic composition. In some embodiments, the surfactant is present in an amount of from about 25% to about 50% by weight of the solid cosmetic composition. In some embodiments, the surfactant is present in an amount of from about 25% to about 40% by weight of the solid cosmetic composition. In some embodiments, the surfactant is present in an amount of from about 25% to about 35% by weight of the solid cosmetic composition. In some embodiments, the surfactant is present in an amount of from about 25% to about 30% by weight of the solid cosmetic composition.

In some embodiments, the surfactant is selected from the group consisting of anionic surfactant, amphoteric surfactant, cationic surfactant, non-ionic surfactant and mixtures thereof.

In some embodiments, the surfactant comprises an anionic surfactant. In some embodiments, the surfactant comprises an anionic surfactant selected from the group consisting of sodium lauryl sulfate, sodium laureth sulfate, sodium dodecyl sulfate, sodium cocosulfate, ammonium lauryl sulfate, ammonium laureth sulfate, sodium myreth sulfate, sodium lauroyl sarcosinate, disodium laureth sulfosuccinate, disodium lauryl sulfosuccinate, sodium lauryl sulfoacetate, sodium cocoyl isethionate, sodium olefin sulfonate, and mixtures thereof. In some embodiments, the surfactant comprises an anionic surfactant selected from the group consisting of sodium lauryl sulfate, sodium laureth sulfate, sodium cocosulfate, and mixtures thereof.

In some embodiments, the surfactant comprises sodium lauryl sulfate. In some embodiments, the surfactant comprises sodium laureth sulfate. In some embodiments, the surfactant comprises sodium cocosulfate.

In some embodiments, the surfactant comprises an amphoteric surfactant. In some embodiments, the surfactant comprises an amphoteric surfactant selected from the group consisting of lauryl betaine, cocamidopropyl betaine, trimethyl glycine betaine, sodium cocoamphoacetate, disodium cocoamphodiacetate, sodium lauroamphoacetate, disodium lauroamphodiacetate, cocoamidopropyl hydroxysultaine, and mixtures thereof. In some embodiments, the surfactant comprises an amphoteric surfactant selected from the group consisting of lauryl betaine, cocamidopropyl betaine, sodium cocoamphoacetate, and mixtures thereof.

In some embodiments, the surfactant comprises a cationic surfactant. In some embodiments, the surfactant comprises a cationic surfactant selected from the group consisting of cetrimonium chloride, behentrimonium chloride, and mixtures thereof.

In some embodiments, the surfactant comprises a combination of an anionic surfactant (such as sodium lauryl sulfate and/or sodium cocosulfate) and a cationic surfactant (such as cetrimonium chloride). In some embodiments, the surfactant comprises a combination of an anionic surfactant (such as sodium lauryl sulfate and/or sodium cocosulfate) and an amphoteric surfactant (such as lauryl betaine).

In some preferred embodiments, the surfactant is provided in solid or essentially anhydrous form. As used herein, “essentially anhydrous surfactant” means a surfactant product comprising water in an amount of less than about 15% by weight of the surfactant product, preferably less than about 10% by weight of the surfactant product. Preferably, the surfactant included in the composition is provided as a surfactant product comprising water in an amount of no greater than about 10% by weight of the surfactant product, preferably no greater than 8% by weight of the surfactant product. The amount of active surfactant in the surfactant product included in the composition is therefore preferably at least 85% by weight of the surfactant product, preferably at least 90% by weight of the surfactant product.

In some embodiments, the surfactant is an essentially anhydrous surfactant selected from the group consisting of sodium lauryl sulfate, sodium laureth sulfate, sodium dodecyl sulfate, sodium cocosulfate, ammonium lauryl sulfate, ammonium laureth sulfate, sodium myreth sulfate, sodium lauroyl sarcosinate, disodium laureth sulfosuccinate, disodium lauryl sulfosuccinate, sodium lauryl sulfoacetate, sodium cocoyl isethionate, sodium olefin sulfonate, lauryl betaine, cocamidopropyl betaine, trimethyl glycine betaine, sodium cocoamphoacetate, disodium cocoamphodiacetate, sodium lauroamphoacetate, disodium lauroamphodiacetate, cocoamidopropyl hydroxysultaine, and mixtures thereof. In some embodiments, the surfactant is an essentially anhydrous surfactant selected from the group consisting of sodium lauryl sulfate, sodium laureth sulfate, sodium dodecyl sulfate, sodium cocosulfate, ammonium lauryl sulfate, ammonium laureth sulfate, sodium myreth sulfate, sodium lauroyl sarcosinate, disodium laureth sulfosuccinate, disodium lauryl sulfosuccinate, sodium lauryl sulfoacetate, sodium cocoyl isethionate, sodium olefin sulfonate, lauryl betaine, cocamidopropyl betaine, trimethyl glycine betaine, sodium cocoamphoacetate, disodium cocoamphodiacetate, sodium lauroamphoacetate, disodium lauroamphodiacetate, cocoamidopropyl hydroxysultaine, cetrimonium chloride, behentrimonium chloride, and mixtures thereof. In some embodiments, the surfactant is an essentially anhydrous surfactant selected from the group consisting of sodium lauryl sulfate, sodium laureth sulfate, sodium cocosulfate, and mixtures thereof. In some embodiments, the surfactant is essentially anhydrous sodium lauryl sulfate. In some embodiments, the surfactant is essentially anhydrous sodium cocosulfate.

### Wax

The solid cosmetic composition comprises a wax. The wax may be any suitable wax for preparing a solid cosmetic composition.

Preferably, the wax is a natural wax, for example, an animal wax, plant wax and/or modified animal or plant wax. In some embodiments, the wax is selected from the group consisting of beeswax, vegetable wax, and mixtures thereof. In some embodiments, the wax is a vegetable wax selected from the group consisting of Japan wax, carnauba wax, rapeseed wax, and mixtures thereof.

In some preferred embodiments, the wax is or comprises a self-emulsifying wax. The use of a self-emulsifying wax is beneficial as it may improve hair conditioning properties of the composition, thus making the composition suitable for use as a solid hair conditioner. In some preferred embodiments, the wax is a self-emulsifying wax. In some embodiments, the wax is glyceryl stearate SE. “Glyceryl Stearate SE” is known in the art to be a self-emulsifying form of glyceryl stearate that also contains a small amount of sodium and/or potassium stearate. Glyceryl stearate SE may be produced by reacting an excess of stearic acid with glycerine. The excess stearic acid may then be reacted with potassium and/or sodium hydroxide yielding a product that contains glyceryl stearate as well as potassium stearate and/or sodium stearate.

It was surprisingly found by the present inventors that, when the composition comprises a self-emulsifying wax, the composition may provide an improved conditioning effect on the skin and/or hair of the user. Without wishing to be bound, it was found that, when the product is used as a shampoo, the self-emulsifying wax emulsifies the water in the hair with the surfactants in the composition to provide an additional conditioning effect on the hair. The composition is thus able to be used as both a shampoo and a conditioner simultaneously. In addition, the inclusion of a self-emulsifying wax was surprisingly found to improve the solid structure of the product without having an adverse effect on the light ‘marshmallow-like’ texture. In this regard, it was found that the product could be cut or broken up into smaller pieces suitable for use, whilst reducing the tendency of the product to crumble.

In some embodiments, the wax is or comprises an emulsifying wax. In some embodiments, the wax is an emulsifying wax. As the skilled person will appreciate, an emulsifying wax is a combination of a wax material and a detergent (or surfactant). As used herein, the term “emulsifying wax” is a mixture comprising (i) a fatty acid, fatty alcohol, fatty acid ester, and mixtures thereof, and (ii) a surfactant. As used herein, the term “Emulsifying wax NF” means an emulsifying wax that conforms to the specifications of the National Formulary, and which comprises a combination of cetearyl alcohol and a polysorbate (a polyoxyethylene derivative of a fatty acid ester of sorbitan).

In some embodiments, the wax is an emulsifying wax selected from the group consisting of cetearyl alcohol (and) a sodium alkyl sulfate, cetearyl alcohol (and) a polysorbate, cetearyl alcohol (and) a polymer of ethylene oxide, cetearyl alcohol (and) ceteareth-20, cetearyl alcohol (and) ceteareth-30, glyceryl stearate (and) a polymer of ethylene oxide, cetearyl olivate (and) sorbitan olivate, ethoxylated cetearyl alcohols, ethoxylated stearyl alcohols, and mixtures thereof.

In some embodiments, the wax is an emulsifying wax is selected from cetearyl alcohol (and) sodium lauryl sulfate, cetearyl alcohol (and) sodium laureth sulfate, cetearyl alcohol (and) sodium cocsulfate, and mixtures thereof. In some embodiments, the wax is cetearyl alcohol (and) sodium lauryl sulfate.

In some embodiments, the wax is emulsifying wax NF. In some embodiments, the wax is cetaryl alcohol (and) polysorbate 60 (Polawax).

In some embodiments, the wax is an emulsifying wax selected from the group consisting of cetearyl alcohol (and) sodium lauryl sulfate, cetearyl alcohol (and) polysorbate 60, cetearyl alcohol (and) ceteareth-20, and mixtures thereof.

In some embodiments, the wax may consist of a self-emulsifying wax. For example, the wax may consist of glyceryl stearate SE.

In some embodiments, the wax may consist of an emulsifying wax. For example, the wax may consist of an emulsifying wax selected from the group consisting of cetearyl alcohol (and) sodium lauryl sulfate, cetearyl alcohol (and) polysorbate 60, cetearyl alcohol (and) ceteareth-20, and mixtures thereof. For example, the wax may consist of cetearyl alcohol (and) sodium lauryl sulfate.

In some embodiments, the wax comprises a combination of a self-emulsifying wax and an emulsifying wax. For example, the wax may comprise a combination of glyceryl stearate SE and cetearyl alcohol (and) sodium lauryl sulfate. In some embodiments, the wax may comprise a self-emulsifying wax (such as glyceryl stearate SE) and an emulsifying wax (such as cetearyl alcohol (and) sodium lauryl sulfate) in a weight ratio of from about 5:1 to about 1:5, such as from about 4:1 to about 1:4, such as from about 3:1 to about 1:3, such as from about 2:1 to about 1:2, such as from about 2:1 to about 1:1. In some embodiments, the wax may comprise a self-emulsifying wax (such as glyceryl stearate SE) and an emulsifying wax (such as cetearyl alcohol (and) sodium lauryl sulfate) in a weight ratio of from about 5:1 to about 1:1.

In some embodiments, the wax (such as an emulsifying wax or self-emulsifying wax) is present in an amount of from about 1% to about 30% by weight of the solid cosmetic composition. In some embodiments, the wax (such as an emulsifying wax or self-emulsifying wax) is present in an amount of from about 5% to about 25% by weight of the solid cosmetic composition. In some embodiments, the wax (such as an emulsifying wax or self-emulsifying wax) is present in an amount of from about 5% to about 20% by weight of the solid cosmetic composition. In some embodiments, the wax (such as an emulsifying wax or self-emulsifying wax) is present in an amount of from about 5% to about 15% by weight of the solid cosmetic composition. In some embodiments, the wax (such as an emulsifying wax or self-emulsifying wax) is present in an amount of from about 10% to about 15% by weight of the solid cosmetic composition. In some embodiments, the wax (such as an emulsifying wax or self-emulsifying wax) is present in an amount of from about 15% to about 20% by weight of the solid cosmetic composition.

It was found by the inventors that the combination of wax with a surfactant and aquafaba enabled the preparation of a composition that is solid, and that maintains its solidity over an extended period of time without collapsing. Where gas bubbles are incorporated into the product, the wax may help to maintain the level of aeration in the product during storage, and may thus help to maintain stability of the product during storage. This improves the ‘marshmallow-like’ texture of the product, and helps to maintain the ‘marshmallow-like’ texture over a period of time during storage.

### Aquafaba

As described herein, the solid cosmetic composition comprises aquafaba. As the skilled person will appreciate, aquafaba (literally ‘water bean’) is the name for the water in which legume seeds, such as chickpeas, have been cooked.

Indeed, aquafaba may be prepared using the following method. First, dried legumes, such as chickpeas, may be combined with water at no greater than room temperature. Preferably, the water combined with the legumes is cold water (for example, at a temperature of less than about 10° C.). The water may be combined with the legumes such that the weight ratio of water to legume is from about 20:1 to about 1:1, such as from about 10:1 to about 3:1, such as about 5:1. The resulting combination may then be left for a period of from about 1 hour to about 24 hours, preferably about 12 hours at about room temperature. The combination may then be boiled and simmered for a period of from about 5 minutes to about 180 minutes, such as from about 30 minutes to about 150 minutes, such as from about 60 minutes to about 120 minutes, such as from about 75 minutes to about 105 minutes. The resulting slurry may then be filtered, and simmered to reduce the brine to the desired amount. The resultant brine is what is referred to herein as ‘aquafaba’.

In some embodiments, the aquafaba is derived from a legume that has been cooked in water according to the method described hereinabove, wherein the weight ratio of legume to water is from about 20:1 to about 1:1, such as from about 10:1 to about 3:1, such as about 5:1.

In some embodiments, the aquafaba is derived from a legume having a protein content of at least 15% by weight of the legume, such as in an amount of from about 20% by weight of the legume, such as in an amount of from about 25% by weight of the legume.

The brine may itself subsequently be dried to form a powdered form of aquafaba. Such powder is the dried form of the liquid remaining from cooking the legumes, such as chickpeas. Providing the aquafaba as a powder can be advantageous as it improves ease of handling, and increases the shelf-life of the aquafaba. To incorporate such powder into the cosmetic compositions, the aquafaba powder may be mixed with water (i.e. reconstituted) to yield liquid aquafaba. In some embodiments, the aquafaba is derived from a legume belonging to the family Fabaceae. The legume may belong to the sub-family Faboideae. In some embodiments, the aquafaba is derived from a legume in the family Fabaceae, wherein the legume is of the genus Cicer, Pisum, Glycine, Lens, Vicia, and mixtures thereof. In some embodiments, the aquafaba is derived from a legume selected from the following species: Cicer arietinum, Pisum sativum, Glycine max, Lens culinaris, Lens esculenta, Lens odemensis, Lens ervoides, Lens nigricans, Lens lamottei, Lens tomentosus, and Vicia faba, and mixtures thereof. In some embodiments, the aquafaba is derived from a legume selected from the following species: Cicer arietinum, Pisum sativum, Glycine max, Lens culinaris, and Vicia faba, and mixtures thereof.

In some embodiments, the aquafaba is derived from a legume selected from the group consisting of chickpea, green split pea, yellow split pea, soybean, lentil, fava bean, and mixtures thereof. As used herein “derived from” in this context means that the aquafaba is prepared by soaking the legume in water and preparing the legume brine according to the method described above. In some embodiments, the aquafaba is derived from chickpea (referred to in the art as ‘chickpea brine’).

In some embodiments, the aquafaba (such as chickpea brine) is present in an amount of from about 5% to about 50% by weight of the solid cosmetic composition. In some embodiments, the aquafaba (such as chickpea brine) is present in an amount of from about 10% to about 50% by weight of the solid cosmetic composition. In some embodiments, the aquafaba (such as chickpea brine) is present in an amount of from about 10% to about 40% by weight of the solid cosmetic composition. In some embodiments, the aquafaba (such as chickpea brine) is present in an amount of from about 10% to about 35% by weight of the solid cosmetic composition. In some embodiments, the aquafaba (such as chickpea brine) is present in an amount of from about 10% to about 30% by weight of the solid cosmetic composition. In some embodiments, the aquafaba (such as chickpea brine) is present in an amount of from about 10% to about 25% by weight of the solid cosmetic composition. In some embodiments, the aquafaba (such as chickpea brine) is present in an amount of from about 10% to about 20% by weight of the solid cosmetic composition. In some embodiments, the aquafaba (such as chickpea brine) is present in an amount of from about 15% to about 20% by weight of the solid cosmetic composition. In some embodiments, the aquafaba (such as chickpea brine) is present in an amount of from about 10% to about 15% by weight of the solid cosmetic composition. In some embodiments, the aquafaba (such as chickpea brine) is present in an amount of from about 12.5% to about 17.5% by weight of the solid cosmetic composition.

As used herein, the amount of aquafaba refers to the amount of brine (i.e. the amount of aquafaba in liquid form). For example, the amount of aquafaba may refer to the amount of liquid aquafaba in the form in which it is in immediately after preparation, or in reconstituted form when powdered aquafaba is mixed with water as described hereinabove. Reference to aquafaba should be taken to refer to the liquid form of aquafaba unless specified otherwise.

In some embodiments, the aquafaba comprises protein in an amount of at least about 0.5% by weight of the aquafaba (or legume brine), such as in an amount of at least about 0.75% by weight of the aquafaba, such as in an amount of at least about 1% by weight of the aquafaba, such as in an amount of at least about 5% by weight of the aquafaba, such as in an amount of at least about 10% by weight of the aquafaba.

In some embodiments, the aquafaba (such as chickpea brine) is present in an amount of from about 5% to about 50% by weight of the solid cosmetic composition, wherein the aquafaba comprises protein in an amount of at least about 1% by weight of the aquafaba. In some embodiments, the aquafaba (such as chickpea brine) is present in an amount of from about 10% to about 50% by weight of the solid cosmetic composition, wherein the aquafaba comprises protein in an amount of at least about 1% by weight of the aquafaba. In some embodiments, the aquafaba (such as chickpea brine) is present in an amount of from about 10% to about 40% by weight of the solid cosmetic composition, wherein the aquafaba comprises protein in an amount of at least about 1% by weight of the aquafaba. In some embodiments, the aquafaba (such as chickpea brine) is present in an amount of from about 10% to about 35% by weight of the solid cosmetic composition, wherein the aquafaba comprises protein in an amount of at least about 1% by weight of the aquafaba. In some embodiments, the aquafaba (such as chickpea brine) is present in an amount of from about 10% to about 30% by weight of the solid cosmetic composition, wherein the aquafaba comprises protein in an amount of at least about 1% by weight of the aquafaba. In some embodiments, the aquafaba (such as chickpea brine) is present in an amount of from about 10% to about 25% by weight of the solid cosmetic composition, wherein the aquafaba comprises protein in an amount of at least about 1% by weight of the aquafaba. In some embodiments, the aquafaba (such as chickpea brine) is present in an amount of from about 10% to about 20% by weight of the solid cosmetic composition, wherein the aquafaba comprises protein in an amount of at least about 1% by weight of the aquafaba. In some embodiments, the aquafaba (such as chickpea brine) is present in an amount of from about 15% to about 20% by weight of the solid cosmetic composition, wherein the aquafaba comprises protein in an amount of at least about 1% by weight of the aquafaba. In some embodiments, the aquafaba (such as chickpea brine) is present in an amount of from about 10% to about 15% by weight of the solid cosmetic composition, wherein the aquafaba comprises protein in an amount of at least about 1% by weight of the aquafaba. In some embodiments, the aquafaba (such as chickpea brine) is present in an amount of from about 12.5% to about 17.5% by weight of the solid cosmetic composition, wherein the aquafaba comprises protein in an amount of at least about 1% by weight of the aquafaba.

It was surprisingly found by the present inventors that the inclusion of aquafaba in a composition comprising a surfactant enables the production of a cosmetic product suitable for washing the hair and skin of a user, which has a texture and structure similar to that of marshmallow. This effect was achievable, whilst also ensuring that the product is suitable for use by vegans as no animal-based product (such as eggs and/or gelatin) was required in order to achieve this effect.

### Acidifying Agent

The solid cosmetic composition may further comprise an acidifying agent. Acids may be used in particular to denature the proteins in the aquafaba. It has been found by the present inventors that such denaturation of the proteins may enhance the stability of the marshmallow cosmetic product. The pH of the product will also be reduced, which may beneficially reduce the chemical charge of the hair. This may be beneficial for applications in which the product is used as a hair shampoo or hair conditioning agent.

In some embodiments, the acidifying agent is an organic acidifying agent. In some embodiments, the acidifying agent is an organic acidifying agent selected from the group consisting of citric acid, acetic acid, formic acid, succinic acid, lactic acid, tartaric acid, cream of tartar, fumaric acid, malic acid, maleic acid, oxalic acid, and mixtures thereof. In some embodiments, the acidifying agent is selected from the group consisting of citric acid, acetic acid, lactic acid, tartaric acid, cream of tartar, and mixtures thereof. In some embodiments, the acidifying agent is selected from the group consisting of citric acid, acetic acid, and mixtures thereof. In some embodiments, the acidifying agent is citric acid. In some embodiments, the acidifying agent is acetic acid.

In some embodiments, the acidifying agent is derived from natural sources. In some embodiments, the acidifying agent is derived from natural sources selected from the group consisting of lemon juice, lemon zest, vinegar, and mixtures thereof. In some embodiments, the acidifying agent is derived from lemon juice, lemon zest, vinegar, or mixtures thereof. In some embodiments, the acidifying agent is derived from lemon juice. In some embodiments, the acidifying agent is derived from vinegar.

In some embodiments, the solid cosmetic composition comprises an acidifying agent (such as citric acid and/or acetic acid) in an amount of from about 0.01% to about 10% by weight of the solid cosmetic composition, such as in an amount of from about 0.1% to about 5% by weight of the solid cosmetic composition, such as in an amount of from about 0.5% to about 2.5% by weight of the solid cosmetic composition, such as in an amount of from about 1% to about 1.5% by weight of the solid cosmetic composition. In some embodiments, the solid cosmetic composition comprises an acidifying agent (such as citric acid and/or acetic acid) in an amount of from about 0.5% to about 2% by weight of the solid cosmetic composition, such as in an amount of from about 0.5% to about 1.5% by weight of the solid cosmetic composition, such as in an amount of approximately 1% by weight of the solid cosmetic composition.

### Thickening Agent

In addition to the above, the solid cosmetic composition may further comprise a thickening agent. A thickening agent may be included to increase the solidity of the composition, for example to provide a product that maintains its shape over a prolonged period of time. Where gas bubbles are incorporated into the product, the thickening agent may help to maintain the level of aeration in the product during storage, and may thus help to maintain stability of the product during storage.

In some embodiments, the solid cosmetic composition further comprises a thickening agent. The thickening agent may be a hydrocolloid. As the skilled person will appreciate, hydrocolloids are a heterogeneous group of long chain polymers characterised by their property of forming viscous dispersions and/or gels when dispersed in water. Some hydrocolloids have the ability to modify the rheology of compositions comprising water. There are two types of hydrocolloid: gelling agents (i.e. those that modify the texture and/or solidity of the composition) and thickening agents (i.e. those that modify the viscosity of the composition). The water-thickening property is common to all hydrocolloids, and thus gelling agents can be regarded as a subset of thickening agents. In some embodiments, the thickening agent is also a gelling agent, such as carrageenan, agar, pectin, a combination of a salt of alginic acid and calcium ions, calcium alginate, gellan gum, methyl cellulose, hydroxypropyl methylcellulose, modified starch, and mixtures thereof. In some embodiments, the thickening agent is a hydrocolloid that modifies the viscosity of the composition but which is not a gelling agent, such as xanthan gum, guar gum, konjac gum, gum tragacanth and gum Arabic.

In some embodiments, the thickening agent is selected from the group consisting of xanthan gum, cationic guar gum, agar, pectin, a salt of alginic acid, carrageenan, and mixtures thereof. The salt of alginic acid may be a salt of a monovalent cation; for example, sodium alginate or potassium alginate. The salt of alginic acid may alternatively or additionally be calcium alginate or ammonium alginate.

In some embodiments, the thickening agent is selected from the group consisting of xanthan gum, cationic guar gum, carrageenan, and mixtures thereof. In some embodiments, the thickening agent is cationic guar gum.

In some embodiments, the thickening agent (such as cationic guar gum) is present in an amount of from about 0.01% to about 10% by weight of the solid cosmetic composition, such as from about 0.1% to about 5% by weight of the solid cosmetic composition, such as from about 0.1% to about 2% by weight of the solid cosmetic composition, such as from about 0.5% to about 1.5% by weight of the solid cosmetic composition.

### Humectant

The solid cosmetic composition may further comprise a humectant. It was found by the present inventors that the inclusion of a humectant provided a solid cosmetic composition with a beneficial feel on the skin and good moisturising properties.

In some embodiments, the humectant is selected from the group consisting of glycerine, propylene glycol, 1,3-propanediol, honey, sorbitol, and mixtures thereof. In some embodiments, the humectant is selected from the group consisting of glycerine, propylene glycol, and mixtures thereof. In some embodiments, the humectant is propylene glycol. In some embodiments, the humectant is glycerine. In some embodiments, the humectant is a mixture of propylene glycol and glycerine.

In some embodiments, the humectant is selected from the group consisting of glycerine, monopropylene glycol, dipropylene glycol, 1,3-propanediol, honey, sorbitol, and mixtures thereof. In some embodiments, the humectant is selected from the group consisting of glycerine, monopropylene glycol, and mixtures thereof. In some embodiments, the humectant is monopropylene glycol. In some embodiments, the humectant is glycerine. In some embodiments, the humectant is a mixture of monopropylene glycol and glycerine.

Preferably, the humectant comprises monopropylene glycol. It was found by the present inventors that the inclusion of monopropylene glycol in particular may improve the aeration and ‘marshmallow-like’ texture of the composition, whilst having a desirable humectant effect on the hair and/or skin of the user.

In some embodiments, the humectant (such as propylene glycol and/or glycerine) is present in an amount of from about 5% to about 30% by weight of the solid cosmetic composition, such as from about 8% to about 25% by weight of the solid cosmetic composition, such as from about 10% to about 20% by weight of the solid cosmetic composition. In some embodiments, the humectant (such as propylene glycol and/or glycerine) is present in an amount of from about 10% to about 15% by weight of the solid cosmetic composition. In some embodiments, the humectant (such as propylene glycol and/or glycerine) is present in an amount of from about 15% to about 20% by weight of the solid cosmetic composition.

In some embodiments, the humectant (such as monopropylene glycol) is present in an amount of from about 5% to about 30% by weight of the solid cosmetic composition, such as from about 8% to about 25% by weight of the solid cosmetic composition, such as from about 10% to about 20% by weight of the solid cosmetic composition. In some embodiments, the humectant (such as monopropylene glycol) is present in an amount of from about 10% to about 15% by weight of the solid cosmetic composition. In some embodiments, the humectant (such as monopropylene glycol) is present in an amount of from about 15% to about 20% by weight of the solid cosmetic composition.

In some embodiments, the humectant is propylene glycol and is present in the solid cosmetic composition in an amount of from about 5% to about 30% by weight of the solid cosmetic composition, such as from about 10% to about 20% by weight of the solid cosmetic composition.

In some embodiments, the humectant is monopropylene glycol and is present in the solid cosmetic composition in an amount of from about 5% to about 30% by weight of the solid cosmetic composition, such as from about 10% to about 20% by weight of the solid cosmetic composition, such as from about 15% to about 20% by weight of the solid cosmetic composition.

In some embodiments, the weight ratio of humectant (such as propylene glycol and/or glycerine) to wax (such as emulsifying wax) is from about 5:1 to about 1:5, such as from about 3:1 to about 1:3, such as from about 2:1 to about 1:2, such as approximately 1:1.

In some embodiments, the weight ratio of humectant (such as monopropylene glycol) to wax (such as self-emulsifying wax) is from about 5:1 to about 1:5, such as from about 3:1 to about 1:3, such as from about 2:1 to about 1:2, such as from about 2:1 to about 1:1, such as approximately 1:1.

In some embodiments, the weight ratio of humectant (such as propylene glycol and/or glycerine) to surfactant (such as sodium lauryl sulfate and/or sodium cocosulfate) is from about 1:1 to about 1:10, such as from about 1:1 to about 1:5, such as from about 1:2 to about 1:4.

In some embodiments, the weight ratio of humectant (such as monopropylene glycol) to surfactant (such as sodium lauryl sulfate and/or sodium cocosulfate) is from about 1:1 to about 1:10, such as from about 1:1 to about 1:5, such as from about 1:1 to about 1:4, such as from about 1:2 to about 1:3. In some embodiments, the weight ratio of humectant (such as monopropylene glycol) to surfactant (such as sodium lauryl sulfate and/or sodium cocosulfate) is from about 1:1 to about 1:10, such as from about 1:1 to about 1:3, such as from about 1:1 to about 1:2.

In some embodiments, the weight ratio of humectant (such as propylene glycol and/or glycerine) to aquafaba (such as chickpea brine) is from about 1:1 to about 1:5, such as from about 1:1 to about 1:2.

In some embodiments, the weight ratio of humectant (such as monopropylene glycol) to aquafaba (such as chickpea brine) is from about 5:1 to about 1:5, such as from about 2:1 to about 1:2, such as from about 2:1 to about 1:1. In some embodiments, the weight ratio of humectant (such as monopropylene glycol) to aquafaba (such as chickpea brine) is from about 1:1 to about 1:5, such as from about 1:1 to about 1:2.

### Further Components

In addition to the above, the cosmetic composition may further comprise one or more cosmetically acceptable additives. The person skilled in the art is aware of a range of cosmetically acceptable additives which are suitable for incorporation into such compositions.

In some embodiments, the one or more cosmetically acceptable additives are selected from a starch, binder, filler, clay, opacifier, UV absorbing material, UV reflecting material, exfoliating material, essential oil, vitamin, perfume, fragrance, colouring, vegetable butter, vegetable oil, honey, fruit, fruit juice, fruit and/or herb extract, protein, decorative item, silica microsphere, hair growth promoting agent (such as amino acids), and mixtures thereof. Preferably, the one or more cosmetically acceptable additives are selected from the group consisting of a binder, filler, opacifier, exfoliating material, vegetable butter, vegetable oil, essential oil, vitamin, perfume, fragrance, colouring, fruit, fruit juice, fruit extract, protein, herb extract, decorative item, silica microsphere and mixtures thereof.

Fragrance may be added to the product to make the experience of using the present composition more pleasant. Combining essential oils such as lavender, chamomile or rose absolute into fragrances for the invention ensures the user has a pleasant experience.

In some embodiments, the cosmetic composition thus further comprises a perfume or a fragrance. The amount of fragrance is preferably from about 0.1% to about 10% by weight of the total composition, such as from about 0.1% to about 5% by weight of the total composition, such as from about 0.5% to about 5% by weight of the total composition, such as from about 1% to about 5% by weight of the total composition.

The essential oils may be selected based on the fragrance desired, skin type to be treated and other effects desired based on the well-known properties of essential oils. The addition of essential oils, when taken into the nose, is known to alter mood. For example, essential oils are known to create effects of drowsiness or stimulating the senses. Many well documented effects can be achieved by the use of essential oils.

In one embodiment, the one or more essential oils present in the product are selected from Tarragon, Lemon myrtle, Jasmin, Ylang ylang, Labdanum, Lemongrass, Rose otto, Grapefruit, Patchouli, Rosemary, Armois, Lemon, Neroli, Sweet violet, Lavender, Orange 50 fold, Vanilla, Peppermint, Benzoin, Hydrangia, Litsea Cubeba, Cardamon, Tonka, and Chamomile blue. In one embodiment, the one or more essential oils present in the product are selected from Tarragon, Lemon myrtle, Labdunum, and Lemon.

Vitamins, particularly B, C and E are very beneficial for the skin. Vitamin rich ingredients such as Wheatgerm oil can also be used to deliver vitamins on to the skin. In a one embodiment, the vitamins are selected from vitamin B, vitamin C, vitamin E and mixtures thereof. It will be appreciated by one skilled in the art that the vitamin may be provided from any suitable source. For example the vitamin(s) may be provided from a synthetic source or from incorporation into the product of a material, such as a natural material, that has a high vitamin content.

In some embodiments, the solid cosmetic composition further comprises a colouring. The colouring may be any suitable pigment and/or dye. In some embodiments, the solid cosmetic composition may comprise a colouring in an amount of from about 0.001% to about 5% by weight of the total composition.

In some embodiments, the solid cosmetic composition further comprises a protein or source of protein. In some embodiments, the solid cosmetic composition further comprises a protein. The protein or source of protein may be selected from the group consisting of tofu, banana, soya, soya lecithin, hydrolysed wheat protein, and mixtures thereof. When present, the protein may be included in the solid cosmetic composition in an amount of from about 0.01% to about 10% by weight of the solid cosmetic composition, such as in an amount of from about 0.1% to about 5% by weight of the solid cosmetic composition, such as in an amount of from about 1% to about 3% by weight of the solid cosmetic composition.

In some embodiments, the solid cosmetic composition further comprises a fruit, fruit juice, and/or fruit extract. The fruit may be any suitable fruit. Preferably, the fruit may be selected from orange, mango, papaya, strawberry, banana, kiwi, apple, cherry, pineapple, raspberry, blueberry, blackberry, peach, nectarine, and mixtures thereof.

### Total Composition

In some embodiments, the weight ratio of wax (such as emulsifying wax or self-emulsifying wax) to surfactant (such as sodium lauryl sulfate and/or sodium cocosulfate) is from about 1:1 to about 1:10, such as from about 1:1 to about 1:5, such as from about 1:2 to about 1:4. In some embodiments, the weight ratio of wax (such as emulsifying wax and/or self-emulsifying wax) to surfactant (such as sodium lauryl sulfate and/or sodium cocosulfate) is from about 1:1 to about 1:2.

In some embodiments, the weight ratio of wax (such as emulsifying wax or self-emulsifying wax) to aquafaba (such as chickpea brine) is from about 5:1 to about 1:10, such as from about 2:1 to about 1:5, such as from about 1:1 to about 1:3, such as from about 1:1 to about 1:2. In some embodiments, the weight ratio of wax (such as emulsifying wax and/or self-emulsifying wax) to aquafaba (such as chickpea brine) is from about 1:1 to about 2:1.

In some embodiments, the weight ratio of aquafaba (such as chickpea brine) to surfactant (such as sodium lauryl sulfate and/or sodium cocosulfate) is from about 1:1 to about 1:10, such as from about 1:1 to about 1:5, such as from about 1:2 to about 1:3. In some embodiments, the weight ratio of aquafaba (such as chickpea brine) to surfactant (such as sodium lauryl sulfate and/or sodium cocosulfate) is from about 1:1 to about 1:2.

In some embodiments, the solid cosmetic composition comprises:
(i) a surfactant in an amount of from about 15% to about 60% by weight of the solid cosmetic composition;
(ii) a wax in an amount of from about 5% to about 20% by weight of the solid cosmetic composition; and
(iii) aquafaba in an amount of from about 10% to about 25% by weight of the solid cosmetic composition.

In some embodiments, the solid cosmetic composition comprises:
(i) a surfactant selected from the group consisting of sodium lauryl sulfate, sodium laureth sulfate, sodium cocosulfate, and mixtures thereof;
(ii) an emulsifying wax; and
(iii) aquafaba derived from chickpea.

In some embodiments, the solid cosmetic composition comprises:
(i) a surfactant selected from the group consisting of sodium lauryl sulfate, sodium laureth sulfate, sodium cocosulfate, and mixtures thereof;
(ii) a self-emulsifying wax; and
(iii) aquafaba derived from chickpea.

In some embodiments, the solid cosmetic composition comprises:
(i) a surfactant in an amount of from about 15% to about 60% by weight of the solid cosmetic composition, wherein the surfactant is selected from the group consisting of sodium lauryl sulfate, sodium laureth sulfate, sodium cocosulfate, and mixtures thereof;
(ii) an emulsifying wax in an amount of from about 5% to about 20% by weight of the solid cosmetic composition; and
(iii) aquafaba derived from chickpea in an amount of from about 10% to about 25% by weight of the solid cosmetic composition.

In some embodiments, the solid cosmetic composition comprises:
(i) a surfactant in an amount of from about 15% to about 60% by weight of the solid cosmetic composition, wherein the surfactant is selected from the group consisting of sodium lauryl sulfate, sodium laureth sulfate, sodium cocosulfate, and mixtures thereof;
(ii) a self-emulsifying wax in an amount of from about 5% to about 20% by weight of the solid cosmetic composition; and
(iii) aquafaba derived from chickpea in an amount of from about 10% to about 25% by weight of the solid cosmetic composition.

In some embodiments, the solid cosmetic composition comprises:
(i) a surfactant;
(ii) a wax;
(iii) aquafaba; and
(iv) acidifying agent.

In some embodiments, the solid cosmetic composition comprises:
(i) a surfactant;
(ii) a wax;
(iii) aquafaba; and
(iv) a thickening agent.

In some embodiments, the solid cosmetic composition comprises:
(i) a surfactant;
(ii) a wax;
(iii) aquafaba; and
(iv) a humectant.

In some embodiments, the solid cosmetic composition comprises:
(i) a surfactant;
(ii) a wax;
(iii) aquafaba;
(iv) acidifying agent;
(v) a thickening agent; and
(vi) a humectant.

In some embodiments, the solid cosmetic composition comprises:
(i) a surfactant in an amount of from about 15% to about 60% by weight of the solid cosmetic composition;
(ii) a wax in an amount of from about 5% to about 20% by weight of the solid cosmetic composition; and
(iii) aquafaba in an amount of from about 10% to about 25% by weight of the solid cosmetic composition;
(iv) an acidifying agent in an amount of from about 0.01% to about 10% by weight of the solid cosmetic composition;
(v) a thickening agent in an amount of from about 0.1% to about 2% by weight of the solid cosmetic composition; and
(vi) a humectant in an amount of from about 5% to about 30% by weight of the solid cosmetic composition.

In some embodiments, the solid cosmetic composition comprises:
(i) a surfactant selected from the group consisting of sodium lauryl sulfate, sodium laureth sulfate, sodium cocosulfate, and mixtures thereof;
(ii) an emulsifying wax; and
(iii) aquafaba derived from chickpea;
(iv) an acidifying agent selected from the group consisting of citric acid, acetic acid, and mixtures thereof;
(v) a thickening agent selected from the group consisting of xanthan gum, cationic guar gum, carrageenan, and mixtures thereof; and
(vi) a humectant selected from the group consisting of glycerine, propylene glycol, and mixtures thereof.

In some embodiments, the solid cosmetic composition comprises:
(i) a surfactant selected from the group consisting of sodium lauryl sulfate, sodium laureth sulfate, sodium cocosulfate, and mixtures thereof;
(ii) a self-emulsifying wax; and
(iii) aquafaba derived from chickpea;
(iv) an acidifying agent selected from the group consisting of citric acid, acetic acid, and mixtures thereof;
(v) a thickening agent selected from the group consisting of xanthan gum, cationic guar gum, carrageenan, and mixtures thereof; and
(vi) monopropylene glycol.

In some embodiments, the solid cosmetic composition comprises:
(i) a surfactant in an amount of from about 15% to about 60% by weight of the solid cosmetic composition, wherein the surfactant is selected from the group consisting of sodium lauryl sulfate, sodium laureth sulfate, sodium cocosulfate, and mixtures thereof;
(ii) an emulsifying wax in an amount of from about 5% to about 20% by weight of the solid cosmetic composition; and
(iii) aquafaba derived from chickpea in an amount of from about 10% to about 25% by weight of the solid cosmetic composition;
(iv) an acidifying agent in an amount of from about 0.01% to about 10% by weight of the solid cosmetic composition, wherein the acidifying agent is selected from the group consisting of citric acid, acetic acid, and mixtures thereof;
(v) a thickening agent in an amount of from about 0.1% to about 2% by weight of the solid cosmetic composition, wherein the thickening agent is selected from the group consisting of xanthan gum, cationic guar gum, carrageenan, and mixtures thereof; and
(vi) a humectant in an amount of from about 5% to about 30% by weight of the solid cosmetic composition, wherein the humectant is selected from the group consisting of glycerine, propylene glycol, and mixtures thereof.

In some embodiments, the solid cosmetic composition comprises:
(i) a surfactant in an amount of from about 15% to about 60% by weight of the solid cosmetic composition, wherein the surfactant is selected from the group consisting of sodium lauryl sulfate, sodium laureth sulfate, sodium cocosulfate, and mixtures thereof;
(ii) a self-emulsifying wax in an amount of from about 5% to about 20% by weight of the solid cosmetic composition; and
(iii) aquafaba derived from chickpea in an amount of from about 10% to about 25% by weight of the solid cosmetic composition;
(iv) an acidifying agent in an amount of from about 0.01% to about 10% by weight of the solid cosmetic composition, wherein the acidifying agent is selected from the group consisting of citric acid, acetic acid, and mixtures thereof;
(v) a thickening agent in an amount of from about 0.1% to about 2% by weight of the solid cosmetic composition, wherein the thickening agent is selected from the group consisting of xanthan gum, cationic guar gum, carrageenan, and mixtures thereof; and
(vi) a humectant in an amount of from about 5% to about 30% by weight of the solid cosmetic composition, wherein the humectant is monopropylene glycol.

In some embodiments, the density of the solid cosmetic composition is less than about 1 g/cm³. Preferably, the density of the solid cosmetic composition is less than about 0.8 g/cm³, such as less than about 0.7 g/cm³, such as less than about 0.6 g/cm³, such as less than about 0.5 g/cm³. Preferably, the density of the solid cosmetic composition is from about 0.1 g/cm³ to about 0.6 g/cm³.

In some embodiments, the solid cosmetic composition further comprises gas bubbles. The gas bubbles may be randomly distributed in the composition. In some embodiments, the solid cosmetic composition has dispersed therein gas bubbles, wherein the gas bubbles form from about 5% to about 90% by volume of the solid cosmetic composition. Preferably, the solid cosmetic composition has dispersed therein gas bubbles, wherein the gas bubbles form from about 20% to about 85% by volume of the solid cosmetic composition, such as from about 40% to about 80% by volume of the solid cosmetic composition, such as from about 50% to about 75% by volume of the solid cosmetic composition. In some embodiments, the solid cosmetic composition has dispersed therein gas bubbles, wherein the gas bubbles form from about 20% to about 50% by volume of the solid cosmetic composition.

In some preferred embodiments, the composition comprises water in a total amount of no greater than about 15% by weight of the solid cosmetic composition, such as in a total amount of no greater than about 10% by weight of the solid cosmetic composition, such as in a total amount of no greater than about 5% by weight of the solid cosmetic composition, such as in an amount of no greater than about 1% by weight of the solid cosmetic composition. As used herein the “total amount” of water includes any water that is provided by the surfactant product included in the composition. The “total amount of water” does not include any water provided by the aquafaba or legume brine.

Preferably, the solid cosmetic composition is suitable for use in washing the hair and/or body of the user. Preferably, the solid cosmetic composition is suitable for use in washing both the hair and body of the user. Preferably, the solid cosmetic composition is also suitable for use in conditioning the hair and/or body of the user. In some preferred embodiments, the solid cosmetic composition is a solid shower gel. In some preferred embodiments, the solid cosmetic composition is a solid shampoo. In some preferred embodiments, the solid cosmetic composition is a “2-in-1” shampoo and conditioner product.

In some preferred embodiments, the solid cosmetic composition is suitable for use by vegan users. In other words, the solid cosmetic composition is preferably entirely free from animal-based products (such as eggs and/or gelatin).

The above ranges provide preferred amounts of each of the components. Each of these ranges may be taken alone or combined with one or more other component ranges to provide a preferred aspect of the invention.

### Process

As described herein, according to a second aspect of the invention, there is provided a process for the production of a solid cosmetic composition as defined herein, the process comprising the steps of:
(a) combining:
   (i) a surfactant;
   (ii) a wax; and
   (iii) aquafaba; and
(b) solidifying the resultant composition.

As used herein, reference to “aquafaba” in the process for preparation of a solid cosmetic composition refers to aquafaba in its liquid form (e.g. immediately after preparation of the aquafaba or after reconstitution of powdered aquafaba).

In some embodiments, step (a) of the process comprises (i) first combining the surfactant and the wax, (ii) combining the aquafaba with the resultant combination of surfactant and wax to form a mixture, and (iii) agitating the mixture. In some embodiments, the aquafaba is itself agitated (such as by whisking) prior to its combination with surfactant and wax. It will be understood by one skilled in the art that step (a)(iii) of agitating the mixture may be performed by any suitable agitation. The agitation can, for example, be mixing, such as high-speed or high-shear mixing. Preferably, the agitation is achieved by whisking. The agitation may preferably incorporate gas bubbles into the mixture; i.e. agitation may aerate the mixture.

Whisking is the preferred method of agitating the mixture of aquafaba, surfactant and wax. If agitation is achieved through use of a whisk, this can be either a hand whisk, an electric hand whisk or industrial Hobart mixer with a whisk attachment. In some embodiments, agitation may be performed by whisking at a low to medium speed (for example, between about 300 to about 1,000 rpm) or at a medium to high speed (for example, between about 1,000 rpm to about 10,000 rpm). The mixture may be whisked at any suitable temperature to maintain aeration. A preferred temperature is from about 20° C. to about 90° C., such as from about 25° C. to about 80° C., such as from about 40° C. to about 70° C.

In some embodiments, the aquafaba is first agitated (such as by whisking) prior to its combination with surfactant and wax. Preferably, the aquafaba may be agitated (such as by whisking) such that the agitated aquafaba that is combined with the surfactant and wax comprises gas bubbles in an amount of at least about 50% by volume of the aquafaba, such as in an amount of at least about 70% by volume of the aquafaba, such as in an amount of at least about 80% by volume of the aquafaba. Preferably, the aquafaba may be agitated (such as by whisking) such that the agitated aquafaba that is combined with the surfactant and wax comprises gas bubbles in an amount of from about 70% to about 90% by volume of the aquafaba.

In some embodiments, the mixture of aquafaba, surfactant and wax is agitated (such as by whisking) such that the resulting solid cosmetic composition comprises gas bubbles dispersed therein in an amount of from 5% to 90% by volume of the solid cosmetic composition, such as from about 20% to about 85% by volume of the solid cosmetic composition, such as from about 40% to about 80% by volume of the solid cosmetic composition, such as from about 50% to about 75% by volume of the solid cosmetic composition.

In some embodiments, the aquafaba is first whisked prior to its combination with surfactant and wax. The aquafaba may be whisked on a medium, medium-high or high speed until it forms stiff peaks prior to its combination with surfactant and wax. The surfactant and wax may then be combined with the aquafaba, whilst continuing to whisk the mixture in order to maintain aeration in the mixture.

In some embodiments, further additives may be added (such as humectant, thickening agent, acidifying agent, fragrance, colouring) during step (a) either at the same time as combining the surfactant, wax and aquafaba, or after these components have been combined. Preferably, any further additives are also agitated together with the surfactant, wax and aquafaba in order to maintain aeration and obtain an acceptable blend of ingredients.

The process described herein may further comprise a preliminary step (0) of preparing the aquafaba before step (a). First, in step (0), water may be combined with dried legumes, such as chickpeas, at no greater than room temperature. Preferably, the water combined with the legumes is cold water (at a temperature of less than about 10° C.). The water may be combined with the legumes such that the weight ratio of water to legume is from about 20:1 to about 1:1, such as from about 10:1 to about 3:1, such as about 5:1. The resulting combination may then be left for a period of from about 1 hour to about 24 hours, preferably about 12 hours at about room temperature. The combination may then be boiled and simmered for a period of from about 5 minutes to about 180 minutes, such as from about 30 minutes to about 150 minutes, such as from about 60 minutes to about 120 minutes, such as from about 75 minutes to about 105 minutes. The resulting slurry may then be filtered, and simmered to reduce the brine to the desired amount of aquafaba.

In some embodiments, the aquafaba may be provided as reconstituted aquafaba that is prepared by mixing powdered aquafaba with water. In such embodiments, the process may comprise the additional step (0)-(i) of reconstituting powdered aquafaba to form aquafaba (i.e. in liquid form).

An exemplary process for preparing a product in accordance with the present invention is as follows:
1. The legume brine is first prepared according to the following steps:
   a. dried legumes (such as chickpeas) are added to tap water (in a weight ratio of from 10:1 to 1:1 legume to water, such as about 5:1), and left to soak for 12 hours;
   b. without discarding any water, the legume/water mixture may then be heated to bring it to the boil, and then left to simmer for 90 minutes;
   c. the mixture may then be filtered;
   d. the filtered mixture may then be simmered to reduce the amount of brine.
2. The surfactant and wax are combined (possibly with a humectant, if used), and heated to a temperature of between 70-90° C. to solubilise the surfactant.
3. The legume brine (together with acidifying agent, if used) may be whisked on a high speed until stiff peaks of legume brine are formed.
4. If using, a thickening agent may then be gradually added to the legume brine mixture whilst whisking on a lower speed (e.g. a medium speed).
5. While the mixture of surfactant and wax (and optionally humectant) is still semi-hot (about 50° C.), this mixture may be added gradually into the legume brine mixture maintaining aeration, whilst continuing to whisk on a medium speed. These components are whisked together for a period of about 1 to 5 minutes.
6. Any additional components may then be added gradually, whilst continuing to whisk the mixture to maintain the aeration.
7. The resulting mixture is poured into a mould, and left to set.

The shape of the solid compositions of the present invention is not limited. It may be that the solid compositions are provided with a shape that would be aesthetically pleasing and/or which aids in the use of the product. For example, it may be that the solid product is produced in such a manner so that it solidifies in a shape which is ergonomically acceptable to the user. Therefore, in some embodiments of the process of the present invention, the mixture of step (a) is pressed into a mould, allowed to solidify, and then turned out to produce the solid product. In some embodiments, the process comprises solidifying the mixture in a rectangular cuboid mould in order to produce a product having a rectangular cuboid shape, which may be cut into slices by the user. Alternatively, or in addition, the mixture may be solidified in moulds having a cube shape such that the resultant products have a shape similar to that of conventional marshmallows.

As described herein, according to a third aspect of the invention, there is also provided a solid cosmetic product obtained or obtainable from the process as described herein.

### Method

As described herein, according to a fourth aspect of the invention, there is provided a cosmetic method comprising contacting the skin or hair of a user with the solid cosmetic composition as defined herein. As will be appreciated by the skilled person, in view of the solid properties of the present invention, the user may contact the skin or hair directly with the solid cosmetic composition. There may be no need to dispense the product from within some external packaging prior to application to the skin, as is the case for conventional liquid shower gel or shampoo products.

### Use

Also described herein is the use of aquafaba for preparing a solid cosmetic composition that has a density of less than about 1 g/cm³. Preferably, the solid cosmetic composition has a texture akin to that of a marshmallow. Preferably, the use of aquafaba is for preparing a vegan solid cosmetic composition.

### EXAMPLES

The invention will now be described with reference to the following non-limiting examples.

### Example 1

A product having the following composition was prepared:

| | Formula | Batch Size (g): |
|---|---|---|
| Component | (% by weight) | 100.00 |
| Propylene Glycol | 15 | 15.00 |
| Polawax | 14 | 14.00 |
| Sodium Lauryl Sulfate (needles) | 40 | 40.00 |
| Chickpea brine (aquafaba) | 15 | 15.00 |
| Lemon juice | 1 | 1.00 |
| Hydrolysed Wheat Protein | 2 | 2.00 |
| Cationic Guar Gum | 1 | 1.00 |
| Fresh fruit (orange, mango, papaya) | 7 | 7.00 |
| Colouring | 2 | 2.00 |
| Fragrance | 3 | 3.00 |
| TOTAL | 100 | 100.00 |

The sodium lauryl sulfate used was Empicol LXV, which is provided in solid needle form with 92% active surfactant and only 8% water.

The product was prepared as follows:
1. The chickpea brine was first prepared according to the following steps:
   a. 15 g dried chickpeas were added to 75 g tap water (weight ratio of 5:1 chickpea to water, and left to soak for 12 hours;
   b. without discarding any water, the chickpea/water mixture was heated to bring it to the boil, and then left to simmer for 90 minutes;
   c. the mixture was then filtered;
   d. the filtered mixture was then simmered to reduce the amount of brine to 15 g.
2. The humectant and wax were combined, and heated to 75° C.
3. The surfactant was then added in, and the heat maintained to solubilise the surfactant needles.
4. The chickpea brine and lemon juice were whisked together on a high speed until stiff peaks of chickpea brine were formed. The whisk speed was then reduced to medium speed, and the hydrolysed wheat protein gradually added in whilst whisking.
5. The cationic guar gum was then gradually added to the chickpea brine and lemon juice mixture whilst whisking on a medium speed.
6. While the mixture of surfactant, humectant and wax is still semi-hot (about 50° C.), this mixture is added gradually into the chickpea brine mixture maintaining aeration, whilst continuing to whisk on a medium speed. These components are whisked together fora period of about 1 to 5 minutes.
7. The remaining components are then added gradually, whilst continuing to whisk the mixture to maintain the aeration.
8. The resulting mixture is poured into a mould, and left to set.

The resulting product was found to have a texture similar to that of marshmallow. The density of the product was very low (less than 1 g/cm³) resulting in a product having a relatively large volume, whilst being very low weight. This improved the ease of transportation, and also provided the user with a more exciting vegan product akin to a marshmallow confection, but without the use of any animal-based ingredients.

### Example 2

A product having the following composition was prepared:

| | Formula | Batch Size (g): |
|---|---|---|
| Component | (% by weight) | 100.00 |
| Monopropylene glycol | 15 | 15.00 |
| Glyceryl Stearate SE | 16 | 16.00 |
| Sodium Lauryl Sulfate (needles) | 36 | 36.00 |
| Chickpea brine (aquafaba) | 16 | 16.00 |
| Lemon juice | 1 | 1.00 |
| Hydrolysed Wheat Protein | 2 | 2.00 |
| Cationic Guar Gum | 1 | 1.00 |
| Treacle | 5 | 5.00 |
| Colouring | 5 | 5.00 |
| Fragrance | 3 | 3.00 |
| TOTAL | 100 | 100.00 |

The sodium lauryl sulfate used was Empicol LXV, which is provided in solid needle form with 92% active surfactant and only 8% water.

The product was prepared as in Example 1. The resulting product is found to have a texture similar to that of marshmallow. The density of the product is very low (less than 1 g/cm³) resulting in a product having a relatively large volume, whilst being very low weight. This improves the ease of transportation, and also provides the user with a more exciting vegan product akin to a marshmallow confection, but without the use of any animal-based ingredients.

### Comparative Example 1

A product having the following composition was prepared:

| | Formula | Batch Size (g): |
|---|---|---|
| Component | (% by weight) | 100.00 |
| Propylene Glycol | 19 | 15.00 |
| Sodium Lauryl Sulfate (needles) | 45 | 40.00 |
| Chickpea brine (aquafaba) | 19 | 15.00 |
| Lemon juice | 1 | 1.00 |
| Cationic Guar Gum | 1 | 1.00 |
| Fresh fruit (orange, mango, papaya) | 10 | 7.00 |
| Colouring | 2 | 2.00 |
| Fragrance | 3 | 3.00 |
| TOTAL | 100 | 100.00 |

The product was prepared as follows:
1. The chickpea brine was prepared according to the method in Example 1.
2. The humectant was heated to 75° C.

Steps 3-8 were followed as in Example 1.

It was found that the product prepared in this comparative example was not sufficiently solid to substantially maintain its shape during storage. The product was too soft, resulting in collapse of the initial marshmallow-like texture, and a less desirable experience for the user.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the claims.

1. A solid cosmetic composition comprising
(i) a surfactant;
(ii) a wax; and
(iii) aquafaba.

2. The solid cosmetic composition according to claim 1, wherein the aquafaba is present in an amount of from about 10% to about 40% by weight of the solid cosmetic composition.

3. The solid cosmetic composition according to claim 1, wherein the aquafaba is present in an amount of from about 10% to about 25% by weight of the solid cosmetic composition.

4. The solid cosmetic composition according to claim 1, wherein the aquafaba is derived from a legume selected from the group consisting of chickpea, green split pea, yellow split pea, soybean, lentil, fava bean and mixtures thereof.

5. The solid cosmetic composition according to claim 1, wherein the aquafaba is derived from chickpea.

6. The solid cosmetic composition according to claim 1, wherein the surfactant is present in an amount of from about 15% to about 60% by weight of the solid cosmetic composition.

7. The solid cosmetic composition according to claim 1, wherein the surfactant is selected from the group consisting of sodium lauryl sulfate, sodium laureth sulfate, sodium dodecyl sulfate, sodium cocosulfate, ammonium lauryl sulfate, ammonium laureth sulfate, sodium myreth sulfate, sodium lauroyl sarcosinate, disodium laureth sulfosuccinate, sodium lauryl sulfoacetate, lauryl betaine, cocamidopropyl betaine, trimethyl glycine betaine, sodium cocoamphoacetate, disodium cocoamphodiacetate, sodium lauroamphoacetate, disodium lauroamphodiacetate, cocoamidopropyl hydroxysultaine, cetrimonium chloride, behentrimonium chloride, and mixtures thereof.

8. The solid cosmetic composition according to claim 1, wherein the wax is present in an amount of from about 5% to about 20% by weight of the solid cosmetic composition.

9. The solid cosmetic composition according to claim 1, wherein the wax comprises a self-emulsifying wax.

10. The solid cosmetic composition according to claim 9, wherein the self-emulsifying wax is glyceryl stearate SE.

11. The solid cosmetic composition according to claim 9, wherein the wax comprises a combination of a self-emulsifying wax and an emulsifying wax in a weight ratio of from about 5:1 to about 1:5.

12. The solid cosmetic composition according to claim 1, further comprising an acidifying agent.

13. The solid cosmetic composition according to claim 12, wherein the acidifying agent is selected from the group consisting of citric acid, acetic acid, lactic acid, tartaric acid, cream of tartar, and mixtures thereof.

14. The solid cosmetic composition according to claim 1, further comprising a thickening agent.

15. The solid cosmetic composition according to claim 14, wherein the thickening agent is selected from the group consisting of xanthan gum, cationic guar gum, agar, pectin, a salt of alginic acid, carrageenan, and mixtures thereof.

16. The solid cosmetic composition according to claim 14, wherein the thickening agent is present in an amount of from about 0.1% to about 2% by weight of the solid cosmetic composition.

17. The solid cosmetic composition according to claim 1, further comprising a humectant.

18. The solid cosmetic composition according to claim 17, wherein the humectant is present in an amount of from about 5% to about 30% by weight of the solid cosmetic composition.

19. The solid cosmetic composition according to claim 17, wherein the humectant is selected from the group consisting of glycerine, monopropylene glycol, dipropylene glycol, 1,3-propanediol, honey, sorbitol, and mixtures thereof.

20. The solid cosmetic composition according to claim 1, further comprising one or more cosmetically acceptable additives selected from a binder, filler, opacifier, exfoliating material, vegetable butter, vegetable oil, essential oil, vitamin, perfume, fragrance, colouring, fruit juice, fruit extract, protein, herb extract, decorative item, silica microsphere and mixtures thereof.

21. A process for the production of a solid cosmetic composition as defined in claim 1, the process comprising the steps of:
(a) combining:
(i) a surfactant;
(ii) a wax; and
(iii) aquafaba; and
(b) solidifying the resultant composition.

22. The process according to claim 21, wherein the step (a) comprises (i) first combining the surfactant and wax, (ii) combining the aquafaba with the resultant combination of surfactant and wax to form a mixture, and (iii) agitating the mixture.

23. The process according to claim 22, wherein the agitation is provided by whisking.

24. A product obtained or obtainable by the process of claim 21.

25. A cosmetic method comprising contacting the skin or hair of a user with the solid cosmetic composition as defined in claim 1.

26. The solid cosmetic composition according to claim 1, wherein the wax comprises an emulsifying wax.
